(19) United States
(12) Patent Application Publication
(10) Pub. No.: US 2007/0135855 A1
(43) Pub. Date: Jun. 14, 2007

(54) Patient management device for portably interfacing with a plurality of implantable medical devices and method thereof

(76) Inventors: Phillip D. Foshee, Woodinville, WA (US); Ralph P. Cardinal, White Bear Lake, MN (US); Bryan Buchanan, Gig Harbor, WA (US); Matthew Fenske, Glen Ellyn, IL (US); Danielle Dahlby McCulloch, Charlotte, NC (US); Brian Lee Robey, Stillwater, MN (US); David C. Johnson, Inver Grove Heights, MN (US)

Correspondence Address: CASCADIA INTELLECTUAL PROPERTY, 500 UNION STREET, STE.1005, SEATTLE, WA, 98101, US

(21) Appl. No.: 11/301,980

(22) Filed: Dec. 13, 2005

(51) Int. Cl. A61N 1/36

(52) U.S. Cl. 607/31; 607/32; 607/60

(57) ABSTRACT

A patient management device for portably interfacing with a plurality of implantable medical devices and method thereof is presented. Permission to interrogate one or more implantable medical devices is authenticated. Patient device data is individually exchanged through interrogation of at least one authenticated implantable medical device through short range telemetry. External device data is exchanged via communication with at least one external device through long range telemetry. At least one of the patient device and external device data is maintained contemporaneously to execution of operations to perform one or more of relay, processing, and outputting of the patient device and external device data subsequent to the interrogation of the implantable medical device.

### FIELD OF THE INVENTION

The invention relates in general to automated patient management and, specifically, to a patient management device for portably interfacing with a plurality of implantable medical devices and method thereof.

### BACKGROUND OF THE INVENTION

Implantable medical devices (IMDs) are fully autonomous therapy delivery and monitoring devices that respectively perform, for example, cardiac pacing, defibrillation, resynchronization, neural stimulation and drug delivery, and physiological data monitoring and collection. IMDs rely on preprogrammed control and can be non-invasively interfaced to external programmers and similar devices, which can interrogate, program, troubleshoot, and download telemetered data through induction or other forms of near-field telemetry.

Currently, IMD interfacing requires an in-clinic visit by the patient once every three to twelve months, or as necessary. Telemetered data downloaded through interrogation is generally analyzed off-line to evaluate patient health status and the telemetered data can include physiological measures available at the time of interrogation, parametric data regarding the status and operational characteristics of the IMD, and observed environmental parameters, such as temperature and time of day. Other types of telemetered data are possible. The frequency and nature of clinical follow-up is dependent upon several factors, including projected battery life, current IMD programming and the need for programming changes, pacing and sensing stability, underlying rhythm or cardiac condition, travel logistics, and the availability of alternative follow-up methods.

Clinical follow-up is conventionally performed through inductive near field telemetry using a programmer under the control of trained healthcare professionals. Identifying significant events that have occurred since the last follow-up session is tied to the frequency of the follow-up sessions, which generally provide the sole opportunity to identify medical problems or concerns. Adhering to a follow-up schedule is crucial, as delays in downloading telemetered data could potentially result in lost data or chronic conditions recognized too late.

Conversely, full access to all data recorded by an IMD is not an absolute prerequisite to following and diagnosing patient well-being. Disease-specific tests typically require only a subset of all available recorded data, which is frequently summarized, for instance, in strip charts, such as electrocardiograms, printed out by conventional programmers for incorporation into hard copy patient medical records. Regardless, the inconvenience and costs of in-clinic visits are nevertheless incurred, even though only partial access to the patient data recorded by IMDs is necessary. An alternative to periodic in-clinic follow-up is needed to facilitate access to IMD-recorded data, including data subsets needed to address areas of possible medical concern, without increasing the attendant burden of conventional IMD interrogation.

In addition to clinical follow-up, downloaded IMD data generally requires processing before being stored into databases in electronic form. Patient data retrieved from each IMD must be individually converted into the specific format used by a particular database, which increases the time and expense required to post-process downloaded patient data. Converting data for an entire patient population can potentially require the expenditure of significant processing and communication resources. However, conventional IMD interrogation devices typically function as download-and-relay conduits that facilitate the retrieval, but forego the processing, of downloaded patient data. Shifting the post-interrogation processing of downloaded patient data to the interrogation device would unburden a centralized repository while taking advantage of untapped resources available on the interrogation device. Conventional approaches, though, refrain from processing downloaded patient data.

U.S. Pat. No. 6,418,346 issued Jul. 9, 2002, to Nelson et al., describes an apparatus and method for remote therapy and diagnosis that includes a personal data manager (PDM) used in a Web-based network. The PDM cooperates with a programmer to remotely monitor IMDs on a chronic basis. The PDM is implemented to store and forward information to personal computers and similar peripheral equipment, or to uplink data from a programmer to a Web-based export data center. The PDM provides a cost-effective extension to the programmer and operates as a data messenger between the programmer, export data center, and IMDs. However, the PMD fails to process downloaded data and is limited to only accessing unregulated non-medical environments on IMDs. Moreover, the PMD does not provide authentication as a precondition to accessing an IMD.

U.S. Pat. No. 6,263,245 issued Jul. 17, 2001, to Snell, describes a system and method for portable implantable device interrogation that can conduct wireless interrogation of an IMD. A portable interrogation device can be directly interfaced with a data processing device, such as a programmer/analyzer. The portable interrogation device includes a control circuit for controlling transmission using telemetry, transmitter for sending signals, receiver for receiving data transmitted by an IMD in response to interrogation signals, memory for storing data received, and electronic communications interface for high-speed delivery of data to the data processing device. However, the device only facilitates relay of data without analysis or processing and fails to provide authentication with IMDs.

Therefore, there is a need for a portable programmer device providing a range of secure functionality, including interrogated patient data processing and analysis, that preferably includes the ability to directly exchange information with a plurality of target devices, including a database, computational or communication device, and hard copy output device.

### SUMMARY OF THE INVENTION

A system and method includes providing a portable patient management device that flexibly interfaces to a plurality of IMDs and various types of distinct external devices. The personal patient management device includes stored credentials to authenticate the device to each IMD and, where required, external device. The device performs a range of functionality that includes functioning as a “surrogate” programmer, relaying patient data retrieved from IMDs as a form of wireless “wand,” converting and formatting patient data for storage into a database, processing the patient data to determine a tangible result for display by the device on a user interface or for use by an external device, and formatting the patient data for output on a hard copy device. In a further embodiment, the device can program IMDs using control parameters either entered directly into the device through the user interface or received from an external device. Other types of functions are possible.

One embodiment provides a patient management device for portably interfacing with a plurality of implantable medical devices and method thereof. Permission to interrogate one or more implantable medical devices is authenticated. Patient device data is individually exchanged through interrogation of at least one authenticated implantable medical device through short range telemetry. External device data is exchanged via communication with at least one external device through long range telemetry. At least one of the patient device and external device data is maintained contemporaneously to execution of operations to perform one or more of relay, processing, and outputting of the patient device and external device data subsequent to the interrogation of the implantable medical device.

A further embodiment provides a patient management device for portably interrogating a plurality of implantable medical devices and method thereof. Permission to interrogate one or more implantable medical devices is authenticated using stored credentials. Patient device data is individually exchanged through interrogation of at least one authenticated implantable medical device through short range telemetry. The patient device is processed contemporaneously to execution of operations to perform one or more of presentation, relay, processing, and outputting of the patient device subsequent to the interrogation of the authenticated implantable medical device

The functionality provided by the portable patient management device enhances the speed and efficiency gains with which patient data is made available to both clinicians and patients. Previously, users often had to wait for cardiac specialists, such as electrophysiologists, and attending physicians to provide patient health status information, where users are now able to retrieve information themselves. Using the portable patient management device, users can receive outputs of either raw or processed patient data faster than current methods available using, for instance, a conventional programmer, due to the user-friendly user interface and the ability to provide direct outputs to external devices, particularly hard copy devices.

Still other embodiments will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a block diagram showing, by way of example, an implantable medical device.

FIG. 2 is a functional block diagram showing, by way of example, a plurality of implantable medical devices in an automated patient management environment.

FIG. 3 is a Venn diagram showing a range of functionality performed by a portable patient management device in the environment of FIG. 2.

FIGS. 4 and 5 are process flow diagrams showing portably interfacing with a plurality of implantable medical devices, in accordance with one embodiment.

FIG. 6 is a process flow diagram showing portably interfacing to a programmer within the processes of FIGS. 4 and 5.

FIG. 7 is a process flow diagram showing portably interfacing to a database within the processes of FIGS. 4 and 5.

FIG. 8 is a process flow diagram showing portably interfacing to a computational system within the processes of FIGS. 4 and 5.

FIG. 9 is a process flow diagram showing portably interfacing to a hard copy device within the processes of FIGS. 4 and 5.

FIG. 10 is a block diagram showing a portable patient management device for portably interfacing with a plurality of implantable medical devices, in accordance with one embodiment.

FIG. 11 is a functional block diagram showing, by way of example, a portable patient management device in handheld factor, in accordance with one embodiment.

### DETAILED DESCRIPTION

Implantable Medical Device

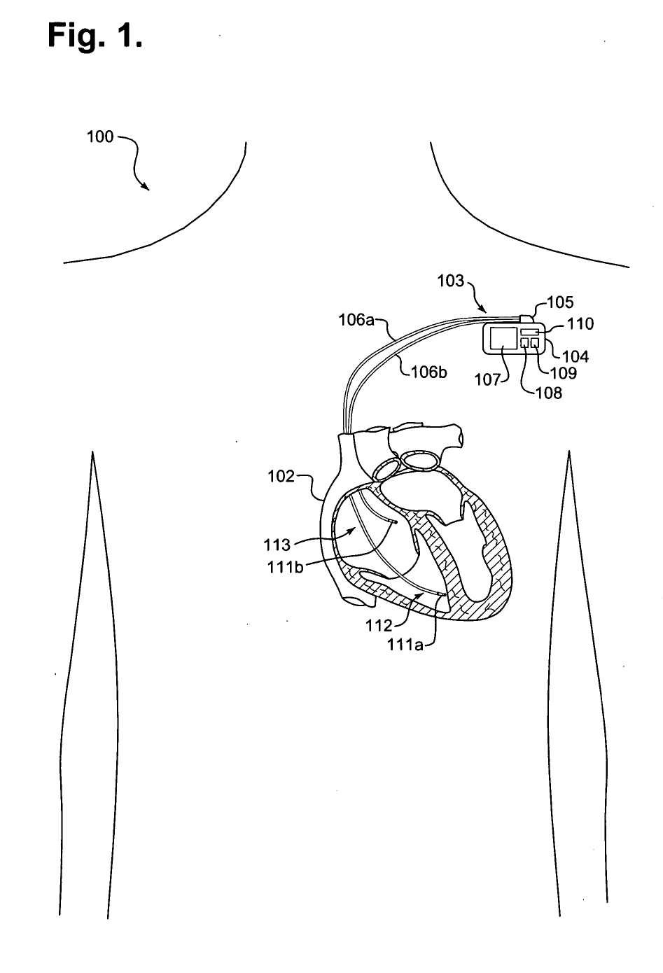

FIG. 1 is a block diagram showing, by way of example, an implantable medical device (IMD) 103. The IMD 103, such as a pacemaker, implantable cardiac defibrillator (ICD) or similar device, is surgically implanted in the chest or abdomen of a patient to provide in situ therapy, such as pacing, cardiac resynchronization, defibrillation, neural stimulation and drug delivery, and physiological data monitoring. Other types of IMDs are possible, including cardiac and other disease-related IMDs and IMDs for other forms of medical therapy and monitoring.

The IMD 103 includes a case 104 and terminal block 105 coupled to a set of leads 106a-b. The leads 106a-b are implanted transvenously for endocardial placement. Other types of leads, such as used with legacy epicardial and subcutaneous systems, are possible. The IMD 103 is in direct electrical communication with the heart 102 through electrodes 111a-b positioned on the distal tips of each lead 106a-b. Other types of electrodes and electrode positioning, such as combinations and permutations of distal and proximal electrodes, are possible. Additionally, other forms and variations of cardiac therapy, including pacing, shocking, and resynchronization, are possible. By way of example, the set of leads 106a-b can include a right ventricular electrode 111a, preferably placed in the right ventricular apex 112 of the heart 102, and a right atrial electrode 111b, preferably placed in the right atrial chamber 113 of the heart 102.

The IMD 103 includes a case 104 and terminal block 105 coupled to a set of leads 106a-b. The IMD case 104 houses hermitically-sealed components, including a battery 107, control circuitry 108, memory 109, and telemetry circuitry 110. The battery 107 provides a finite, power source. The control circuitry 108 controls therapy delivery and monitoring, including the delivery of electrical impulses to the heart 102 and sensing of spontaneous electrical activity. The memory 109 includes a memory store in which the physiological signals sensed by the control circuitry 108 can be temporarily stored, pending telemetered data download.

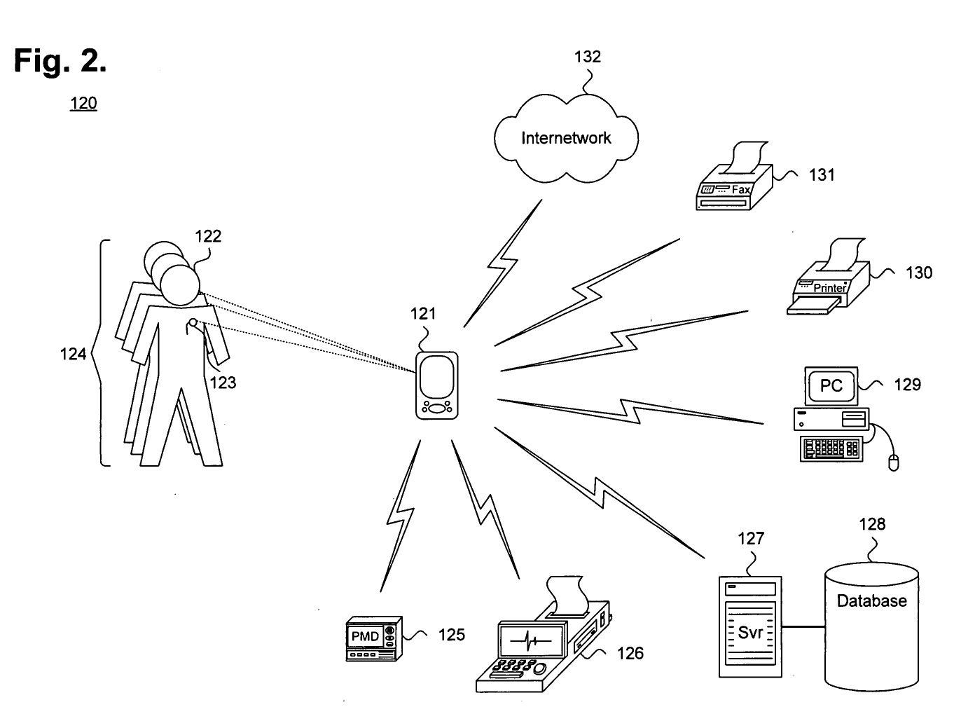

The telemetry circuitry 110 provides an interface between the IMD 103 and an external device, such as a stationary patient management device (PMD), conventional programmer, ambulatory repeater, such as described in commonly-assigned U.S. patent application Ser. No. 11/113,206, filed Apr. 22, 2005, pending, the disclosure of which is incorporated by reference, or similar device, as well as a portable patient management device, as further described below beginning with reference to FIG. 2. The portable patient management device can serve as a form of wireless wand to facilitate the interrogation of the IMD 103 using a convenient handheld form factor, either in-clinic or at large, in addition to providing the ability to directly exchange information with or output information to various target external devices, including a PMD, programmer, database, computational or communications device, and hard copy output device. For near field data exchange, the IMD 103 communicates through inductive telemetry signals exchanged through a wand placed over the location of the IMD 103, via radio frequency (RF) telemetry through a built-in antenna, or other short range wireless means. Programming or interrogating instructions are sent to the IMD 103 and the stored physiological signals are downloaded. For far field data exchange, the IMD 103 communicates through wireless means, such as RF telemetry, with an external device capable of far field telemetry. Other types of wired and wireless data interfaces are possible.

Other configurations and arrangements of leads and electrodes can also be used. Furthermore, although described with reference to IMDs for providing cardiac monitoring and therapy delivery, suitable IMDs also include other types of implantable therapeutic and monitoring devices in addition to or in lieu of cardiac monitoring and therapy delivery IMDs, including IMDs for providing neural stimulation, drug delivery, and physiological monitoring and collection.

Automated Patient Management Environment

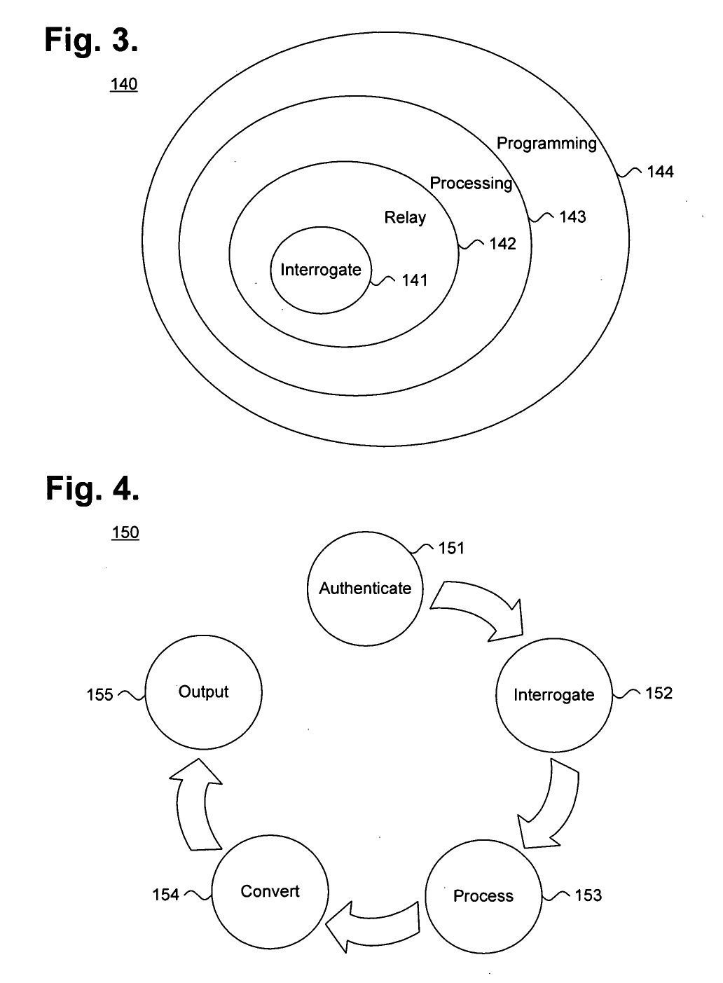

Automated patient management encompasses a range of activities, including remote patient management and automatic diagnosis of patient health, such as described in commonly-assigned U.S. Patent application Pub. No. US2004/0103001, published May 27, 2004, pending, the disclosure of which is incorporated by reference. Such activities can be performed proximal to a patient, such as in the patient's home or office, centrally through a centralized server, such from a hospital, clinic or physician's office, or through a remote workstation, such as a secure wireless mobile computing device. FIG. 2 is a functional block diagram showing, by way of example, a plurality of implantable medical devices in an automated patient management environment 120. In one embodiment, a plurality of patients 124 are proximal to at least one portable patient management device 121. The portable patient management device 121 maintains a set of stored credentials that enables the device to authenticate permission to access and interrogate an IMD 123, such as further described below with reference to FIG. 4. In addition to IMD interrogation, the portable patient management device 121 performs a range of functions, as further described below with reference to FIG. 3, that allow the device to function as a form of “surrogate” programmer and to interact with a range of external devices. The devices include a dedicated patient management device 125, programmer 126, database 128 coupled to a database server 127, personal computer 129, printer 130, facsimile machine 131, and a portal onto an internetwork 132, such as the Internet. Other external devices are possible.

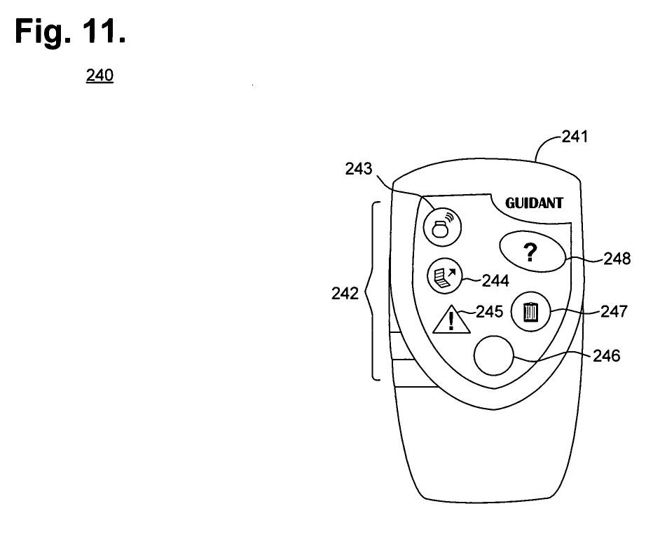

When functioning as a surrogate programmer, the portable patient management device 121 provides full or partial set of core analysis and evaluation functionality, similar to a conventional programmer 126, such as used in clinical practice. However, the portable patient management device 121 lacks the programmer's built-in graphical display and hard copy printer and instead utilizes the external devices 125-132 for providing patient data to clinicians and other users, such as patients. In addition, the portable patient management device 121 is relatively more affordable and accessible to a wider user base due to the lower cost form factor and user-friendly user interface, as further described below with reference to FIG. 11, thereby providing speed and efficiency gains with which patient data is made available. In one embodiment, patient data, in raw or processed form, can be output. By forwarding the output patient data via an external device 125-132, the portable patient management device 121 can also serve as a conduit to referring clinicians and other individuals lacking physical access to the patient. As well, the portable patient management device 121 can be tailored to a specific physician practice or specialty, such as by offering different or customized features sets for cardiologists, heart failure specialists, internists, and electrophysiologists. In a further embodiment, the portable patient management device 121 generates a summary of patient information that is relevant to various clinician contexts. Other forms of surrogate programmer functionality are possible.

With interfacing to a dedicated patient management device 125 or programmer 126, the portable patient management device 121 serves as a form of wireless wand that relays patient data, including physiological measures, parametric data, and environmental parameters, from and to the IMDs 123, such as further described below with reference to FIG. 6. The portable patient management device 121 can be used, for instance, as a form of shared ambulatory programmer to portably interrogate a plurality of IMDs 123, such as in a clinical setting, for later download to a patient management device 125 or programmer 126. In a further embodiment, the portable patient management device 121 can be used to program a plurality of IMDs 123, either in conjunction with or independently from a dedicated patient management device 125 or programmer 126. Other forms of programmer interfacing are possible.

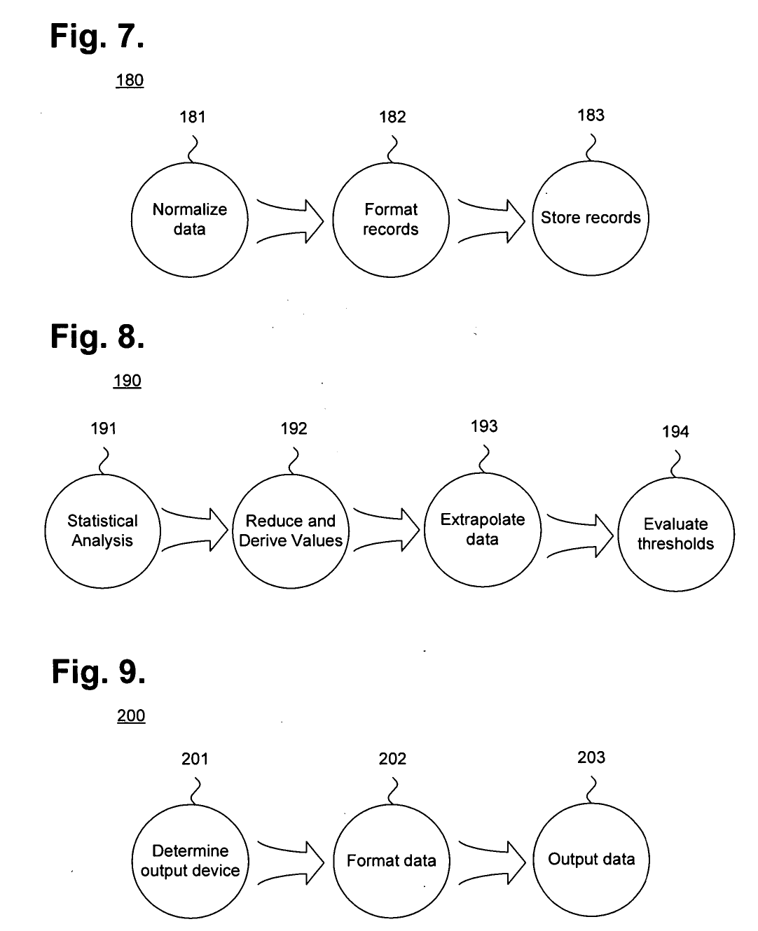

When interfacing to a database 128 via a database server 127, the portable patient management device 121 processes patient data downloaded from IMDs 123 for storage in patient medical records in the database 128, as further described below with reference to FIG. 7. The processing can include normalization and structuring of the data into a format compatible with the database schema. In a further embodiment, the portable patient management device 121 receives data, including partial or complete patient medical records, from the database 128 via the database server 127 for relay to the IMDs 123. Other forms of database processing are possible.

When interfacing to a personal computer 129, or to a centralized server (not shown), the portable patient management device 121 analyzes and evaluates the patient data downloaded from the IMDs 123 for use by the personal computer 129 or dedicated server, as further described below with reference to FIG. 8. In a further embodiment, the portable patient management device 121 can perform analysis and evaluation of the downloaded patient data independently from any interfacing to an external device, such as a personal computer 129 or centralized server, for display or output to the user. The analysis and evaluation can include statistical analysis, value reduction and derivation, data extrapolation, and threshold evaluation. Other forms of analysis and evaluation are possible.

When interfacing to a printer 130, or facsimile machine 131, the portable patient management device 121 facilitates output of processed patient data in hard copy format, as further described below with reference to FIG. 9. In a further embodiment, the processed patient data can be output in electronic format, either in addition to or in lieu of hard copy format, for use by a personal computer 129 or centralized server. The downloaded patient data can be analyzed and formatted into summarized or detailed compilations, including report and spreadsheet formats, for output by the printer 130 or facsimile machine 131, and, in a further embodiment, for use by the personal computer 129 or centralized server. In a still further embodiment, the portable patient management device 121 can receive data from a personal computer 129 or centralized server for processing and, optionally, download to the IMDs 123. Other forms of interaction with hard copy output devices and computational systems are possible.

Finally, when interfacing with a portal to an internetwork 132, the portable patient management device 121 functions as a communications conduit between the IMDs 123 and an external device (not shown) interfaced to the internetwork 132. The internetwork 11 can provide both conventional wired and wireless interconnectivity between the portable patient management device 121 and the external device. In one embodiment, the internetwork 11 is based on the Transmission Control Protocol/Internet Protocol (TCP/IP) network communication specification, although other types or combination of networking implementations are possible. In a manner similar to dedicated patient management device and programmer interfacing, the portable patient management device 121 relays patient data downloaded from the IMDs 123 over the internetwork 132 and, in further embodiment, receives data for processing and, in a still further embodiment, download to the IMDs 123. The portable patient management device 121 can also process the downloaded patient data in a manner similar to personal computer or centralized server interfacing. Other forms of interaction with external communications devices, including internetwork portals, are possible.

Each portable patient management device 121 maintains stored credentials uniquely assigned to each IMD 123 that allows the portable patient management device 121 to be authenticated prior to interrogation, thereby ensuring a secure and legitimate interface to the IMD 123. Each portable patient management device 121 interfaces directly with the external devices 125-132 either through direct means, such as wired connectivity, or through indirect means, such as through inductive telemetry, or via RF or wireless telemetry based on, for example, “strong” Bluetooth, IEEE 802.11 wireless fidelity “WiFi” and “WiMax” interfacing standards. Each portable patient management device 121 could also interface through cellular communications using, for example, CDMA, GSM, GPRS, and WCDMA, compliant protocols, such as described in commonly-assigned U.S. patent application Ser. No. 10/859,649, filed Jun. 3, 2004, pending, the disclosure of which is incorporated by reference. Other forms of wired and wireless interfacing are possible.

Patient data includes physiological measures, which can be quantitative or qualitative, parametric data regarding the status and operational characteristics of the IMD, and environmental parameters, such as the temperature and time of day. Other types of patient data are possible.

In addition, other devices that serve as sources of patient data that collect and forward patient data either as a primary or supplemental function are possible. Additional patient data source devices include, by way of example, medical therapy devices that deliver or provide therapy to the patient 14, medical sensors that sense physiological data in relation to the patient 14, and measurement devices that measure environmental parameters occurring independent of the patient 14. Each patient data source can generate one or more types of patient data and can incorporate one or more components for delivering therapy, sensing physiological data, measuring environmental parameters, or a combination of functionality. In a further embodiment, data values can be entered by a patient 14 directly into a patient data source. For example, answers to health questions could be input into a measurement device that includes interactive user interfacing means, such as a keyboard, display, microphone, and speaker. Such patient-provided data values could also be collected as patient information. Additionally, measurement devices are frequently incorporated into medical therapy devices and medical sensors. Medical therapy devices include implantable medical devices (IMDs), such as pacemakers, implantable cardiac defibrillators (ICDs), cardiac resynchronizers, drug pumps, and neuro-stimulators, and external medical devices (EMDs), such as automatic external defibrillators (AEDs). Medical sensors include implantable sensors, such as implantable heart and respiratory monitors and implantable diagnostic multi-sensor non-therapeutic devices, and external sensors, such as Holter monitors, weight scales, and blood pressure cuffs. Other types of medical therapy, medical sensing, and measuring devices, both implantable and external, are possible.

In a further embodiment, collected patient data can be accessed and analyzed by one or more clients, either locally-configured or remotely-interconnected. The clients can be used, for example, by clinicians to securely access stored patient data assembled in the database 128 or other repository and to select and prioritize patients for health care provisioning, such as respectively described in commonly-assigned U.S. patent application Ser. No. 11/121,593, filed May 3, 2005, pending, and U.S. patent application Ser. No. 11/121,594, filed May 3, 2005, pending, the disclosures of which are incorporated by reference. Although described herein with reference to physicians or clinicians, the entire discussion applies equally to organizations, including hospitals, clinics, and laboratories, and other individuals or interests, such as researchers, scientists, universities, and governmental agencies, seeking access to the patient data.

The collected patient data can also be evaluated for the occurrence of one or more conditions, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 2, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference.

In a still further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

Preferably, the database server 127 is a server-grade computing platform configured as a uni-, multi- or distributed processing system, and the clients are general-purpose computing workstations, such as a personal desktop or notebook computer. In addition, the patient management device 125, database server 127, personal computers 129 and clients are programmable computing devices that respectively execute software programs and include components conventionally found in computing device, such as, for example, a central processing unit (CPU), memory, network interface, persistent storage, and various components for interconnecting these components.

Functionality Performance Range

The portable patient management device 121 is implemented in a portable handheld form factor, as further described below with reference to FIG. 11, and is implemented to perform a range of functionality for interfacing with one or more of the external devices 125-132. FIG. 3 is a Venn diagram showing a range of functionality 140 performed by a portable patient management device 121 in the environment 120 of FIG. 2. The types of functionality provided can be loosely grouped into functions performed to serve as a surrogate programmer 141, relay patient data 143, process or output patient data 144, and, in a further embodiment, program IMDs 144. The groupings of functionality are not discrete and various aspects of relay, process, and program subfunctionality may overlap. Additionally, the functionality groupings are neither prerequisites for nor necessarily dependent upon the other groupings.

Fundamentally, the portable patient management device 121 can serve as a surrogate programmer 141 that provides full or partial set of core analysis and evaluation functionality without built-in detailed output features. The patient data in raw or processed form, is instead forwarded to the external devices 125-132 for output to primary or referring clinicians or to the patient. In a further embodiment, the portable patient management device 121 can be tailored to a specific physician practice or specialty and can also be configured to generate a summary of patient information that is relevant to various clinician contexts.

When providing patient data relay 142, the portable patient management device 121 operates as a form of wireless wand that can interrogate one or more IMDs 123 for data exchange with a stationary PMD 125 or, more conventionally, a programmer 126, or similar device, as further described below with reference to FIG. 6. Patient data relay 142 facilitates efficient in-clinic follow-up that enables patients to be seen without limiting interrogation to examination rooms having, for instance, an available programmer 126. In addition, patient data relay 142 allows patients to perform self-interrogation at large, that is, outside of a clinic, such as at home, to improve the timeliness and ease of IMD interrogation as an adjunct to clinical follow-up.

When providing patient data processing 143, the portable patient management device 121 analyzes and evaluates downloaded patient data to effect a change in form or structure or to generate a tangible result, such as determining a patient health status. Other types of processing are possible. Patient data processing 143 advantageously harnesses the heretofore untapped processing and storage resources available in the patient management-type devices and thereby decreases the burden on computational, communication, and storage resources that otherwise taxes the external devices and infrastructure.

Finally, when performing IMD programming, the portable patient management device 121 becomes an active medical therapy dispensing device that can modify the performance parameters of an IMD, either independently from or in collaboration with an external device, such as a personal computer 129 or centralized server. IMD programming 144 provides advantages similar to patient data relay 142 with increased capabilities. As necessary, IMD programming is generally provided only in response to prescriptive instructions from a qualified healthcare provider. In a still further embodiment, changes to the control parameters of IMDs 123 are determined autonomously, either by the portable patient management device 121 or by an external device. Other types and groupings of portable patient management device functionality are possible.

Device Configuration

The portable patient management device 121 must first be configured prior to interfacing to IMDs and external devices. In one embodiment, the device allows configuration, by way of example, of the following items:
(1) Printer Selection: requires specifying BlueTooth address of supported printers.
(2) User Language Selection: the device provides voice outputs that can be in specified in various languages, such as English, German, French, Spanish, Portuguese, and Italian.
(3) Software Updates: allows updating of device via Bluetooth device interface, which requires a specialized software upgrade device.
(4) Pairing with IMD: specifies supported IMDs and type of short range telemetry used, for instance, heart failure devices with inductive telemetry.
(5) Manufacturing Level Configuration: allows updating of device via Bluetooth device interface, which requires a specialized manufacturing BlueTooth interface device. This configuration allows factory setup of internal software, including updates to IMD protocols and other interface communications.
   Other device configurations are possible. In one embodiment, the configuration sequences can be initialized by a user selecting a series of buttons and the status of the configuration is confirmed by a voice output and tones or beeps.
   Portable Interfacing Process Flow

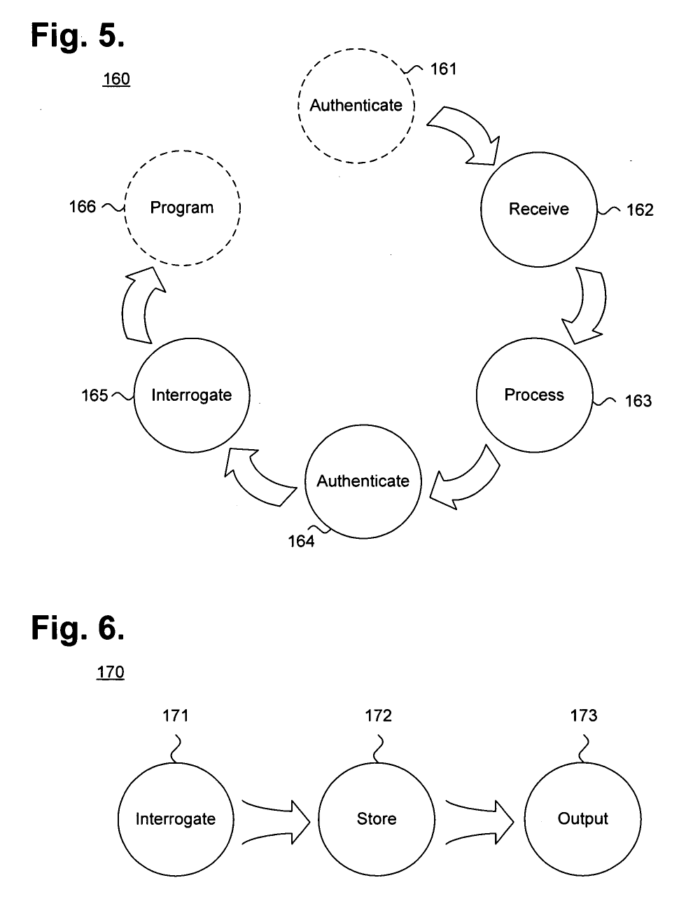

The processes followed when interfacing between an IMD 123 and external devices 125-132 depend upon the type of functions being performed. FIGS. 4 and 5 are process flow diagrams showing portably interfacing with a plurality of implantable medical devices 150, 160, in accordance with one embodiment. Each portable patient management device 121 performs a set of operations common to most types of interfacing, including authentication and interrogation. Patient device-oriented data originates at or is sent to an IMD. External device-oriented data originates at or is sent to external device 125-132. External device-specific processes are described in further detail below with reference to FIGS. 6-9.

Referring first to FIG. 4, a process flow for patient data originating from an IMD 150 is shown. To ensure patient privacy, the portable patient management device 121 must first authenticate (operation 151) permission to access the IMD 123 by providing acceptable credentials, such as described in commonly-assigned U.S. patent application Ser. No. 10/800,806, filed Mar. 15, 2004, pending, the disclosure of which is incorporated by reference. Authentication can include encryption, decryption, certification, authentication, compression, and decompression. In one embodiment, the credentials can include digital certificates, such as an X.509 V3 digital certificate, and public/private and symmetric cryptographic keys using, for instance, special HMAC or other shared secret security mechanisms. Digital certificates and cryptographic keys are further described in R. Orfali et al., “Client/Server Survival Guide,” pp. 147-156, Wiley Comp. Pub. (3d ed. 1999), the disclosure of which is incorporated by reference. In addition, the model numbers and serial numbers of the IMDs can be used for authentication, which can be specified through the Pairing with IMD configuration, such as described above with reference to FIGS. 4 and 5. Other forms of authentication are possible.

In one embodiment, each portable patient management device 121 uses security during printing operations and software upgrades. 128-bit symmetric cryptographic keys are used during printing operations to encrypt and transfer sensitive information to a printer over a secure Bluetooth link using the SAFTER+ symmetric key cryptography. Additionally, each portable patient management device 121 employs “pairing” with user feedback and control to ensure that the device connects and transfers sensitive patient information to the correct printer, as more than one Bluetooth-enabled printer may be in range at any given time. Each portable patient management device 121 uses a trusted software distribution methodology during a software upgrade that requires the software upgrade image to be digitally signed and verified before being installed in a device using 2048-bit asymmetric RSA keys with an SHA-1 hashing algorithm. Other forms of security are possible.

Upon successful authentication, the portable patient management device 121 interrogates (operation 152) the IMD 123 to download stored patient data, which the portable patient management device 121 can then process (operation 153) and convert (operation 154), as necessary, prior to providing an output (operation 155) of the patient data to a receiving external device 125-132. In a further embodiment, the portable patient management device 121 can display the patient data, either in raw or processed form, independently of or in addition to providing an output. Processing of the downloaded patient data involves substantive analysis or evaluation to determine a tangible result, while conversion of the downloaded patient data effects a change in form or structure. The type of processing and conversion performed depends upon the external device destination of the patient data, as further described below with reference to FIGS. 6-9. Other operations on IMD-originated patient data are possible.

Referring next to FIG. 5, a process flow for patient data originating from an external device 160 is shown. Whereas, authentication is a prerequisite to interfacing with an IMD 123, the portable patient management device 121 only need authenticate (operation 161) if the particular external device requires authentication. Generally, external devices that store patient data, either transiently or persistently, will require authentication to ensure patient privacy, while external devices that only output patient data in a physical form without any storage, or which rely on secondary protection of the patient data, such as through encryption or password protection, do not require authentication. External devices that transiently or persistently store patient data include dedicated patient management devices 125, programmers 126, databases 128, personal computers 129, centralized servers, and the infrastructure of an internetwork 132. Other external devices that store patient data are possible. External devices that strictly output and do not store patient data include printers 130 and facsimile machines 131. Other types of external devices that do not store patient data are possible.

Following successfully authenticating, if applicable, the portable patient management device 121 receives (operation 162) incoming patient data and processes (operation 163) the patient data, as necessary. Processing can include determining a tangible result or transforming the incoming data in structure or form. The portable patient management device 121 then authenticates (operation 164) and interrogates (block 165) the IMD 123 to which the incoming patient data applies. In a further embodiment, the portable patient management device 121 programs (operation 166) the IMD 123 through modifying the control parameters. In a further embodiment, the portable patient management device 121 programs the IMD 123 independently or in conjunction with an external device. Other operations on external device-originated patient data are possible.

The particular functions performed by the portable patient management device 121 for various types of external devices will now be described.

Programmer Interfacing

FIG. 6 is a process flow diagram showing portably interfacing to a programmer or a dedicated patient management device 170 within the processes of FIGS. 4 and 5. Upon successful authentication, the portable patient management device interrogates (operation 171) an IMD 123 through either or a combination of near field and far field telemetry. During interrogation, the portable IMD 123 retrieves patient data recorded by and transiently stored on the IMD 123, which is then stored (operation 172) by the portable patient management device 121 until subsequently output (operation 173) to an external device, such as a programmer 126. In a further embodiment, the retrieved patient data can be displayed by the portable patient management device 121. The retrieved patient data can be stored either individually or in combination with patient data retrieved from other IMDs 123 as separately-identifiable data sets. Additionally, the portable patient management device 121 can output all or some of the patient data sets to the external device and the data sets can be either deleted or persistently maintained on the portable patient management device 121 following output or display. Other types of interfacing to a programmer 126 or dedicated patient management device 125 are possible.

Database Interfacing

FIG. 7 is a process flow diagram showing portably interfacing to a database 180 within the processes of FIGS. 4 and 5. Following successful authentication, interrogation, and patient data download and storage, as further described above with reference to FIG. 6, the portable patient management device 121 normalizes the received patient data (operation 161), if necessary, to convert the patient data into a form suitable for storage in the database 128. For instance, intrathoracic impedance values might be converted into intracardial pressure measures that are independent of the particular physiology exhibited by the patient. The portable patient management device 121 then formats the patient data into records (operation 162), which are stored (operation 163) into the database 128 via the database server 127. The record formatting can also be performed in combination with the database server 127. Other types of interfacing to a database are possible.

Computational System Interfacing

FIG. 8 is a process flow diagram showing portably interfacing to a computational system 190 within the processes of FIGS. 4 and 5. Following successful authentication, interrogation, and patient data download and storage, as further described above with reference to FIG. 6, the portable patient management device 121 can formulate tangible results from the retrieved patient data through one or more methods. For instance, the patient data could undergo statistical analysis (operation 171) to recognize trends indicating an onset, progression, regression, absence, or status quo of one or more health conditions. The patient data could also be reduced and have further values derived (operation 172) or extrapolated (operation 173). Commonly, patient data can be evaluated against one or more thresholds (operation 174) to facilitate identifying patient physiological aspects whose profiles have changed significantly enough to warrant further consideration. Threshold evaluation can include enumerating notifications of thresholds being exceeded, such as described in commonly-assigned U.S. patent application Ser. No. 11/121,870, filed May 3, 2005, pending, the disclosure of which is incorporated by reference. For instance, in one embodiment, where a IMD 123 can denote errors or warnings, the portable patient management device 121 decodes the errors or warnings, which are annunciated to a clinician through external device 125-132 or, in a further embodiment, via a user interface provided by the portable patient management device 211. A programmer 126 or personal computer 129, for example, could be configured to flag the error or warning and a printer 130 or facsimile machine 131 could automatically generate a report or send a facsimile detailing the error or warning to the clinician. Other threshold notification enumerations are possible.

Following processing, the patient data can be output to a computational system, such as a personal computer 129, centralized server, or an external device interfaced through the internetwork 132. In a further embodiment, the processed patient data can be displayed by the portable patient management device 121. Other forms of portable interfacing to a computational system are possible.

Hard Copy Device Interfacing

FIG. 9 is a process flow diagram showing portably interfacing to a hard copy device 200 within the processes of FIGS. 4 and 5. Following successful authentication, interrogation, and patient data download and storage, as further described above with reference to FIG. 6, the portable patient management device 121 first determines the type of output device (operation 181) to which the retrieved patient data will be sent, such as a printer 130 or facsimile machine 131. The patient data is then formatted for (operation 182) and output to (operation 183) the destination output device. Data formatting may involve structuring the patient data into a report format and can also include processing of the data, such as described above with reference to FIG. 8. Other types of portable interfacing to a hard copy device are possible.

Patient Management Device

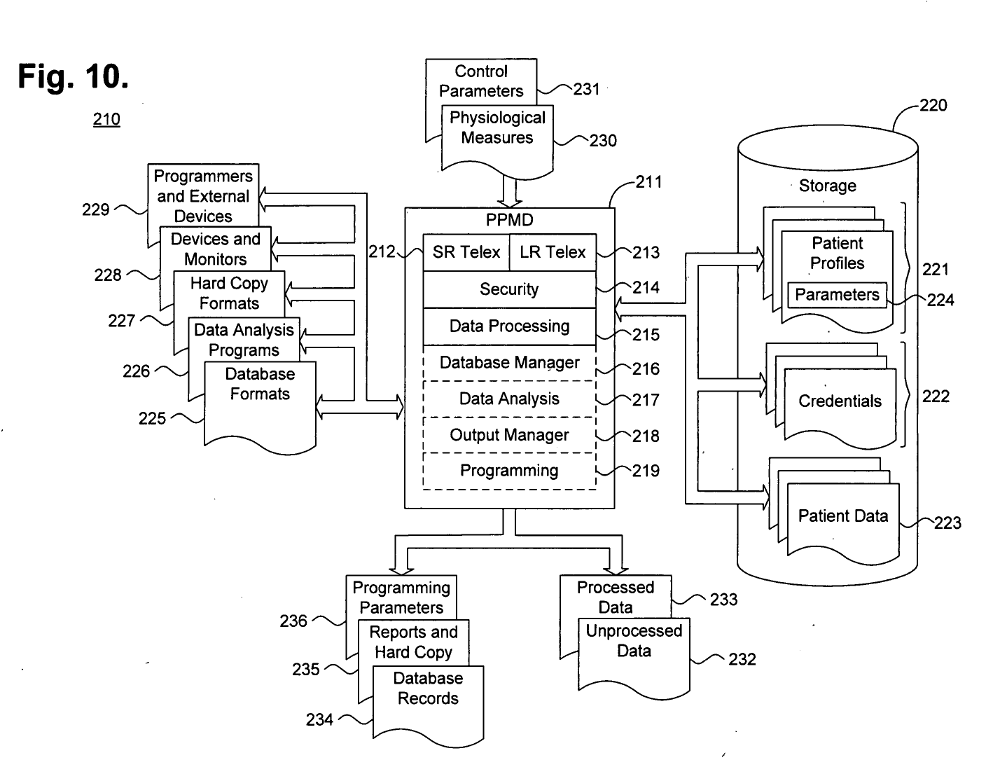

The core analysis and evaluation functionality provided by the portable patient management device can be a full or partial set of operations available on a conventional programmer without the built-in output devices. Further, the types of interfacing functions performed by a portable patient management device depend upon the type of external device to which the portable patient management device is configured to implement. Consequently, some or all of the functionality required to interface to the various types of external devices 125-132 may be present. FIG. 10 is a block diagram showing a portable patient management device 211 for portably interfacing with a plurality of implantable medical devices 210, in accordance with one embodiment. The portable patient management device 211 executes a sequence of programmed process steps, such as described above with reference to FIGS. 4-9, implemented, for instance, on a special purpose programmed digital computer platform or embedded system.

The portable patient management device 211 includes storage 220, which maintains patient profiles 221, and credentials 222 for authenticating the device to IMDs and external devices that require authentication, and patient data 223. The patient profiles 211 include parameters 224 that, in a further embodiment, control the therapy provided by IMDs. The portable patient management device 211 also includes volatile memory for providing program and data stores and non-volatile for storing configuration settings, such as described above with reference to FIGS. 4 and 5, and other device data that may require persistent storage. Other types of information can be stored in the storage 220 and memories.

The portable patient management device 211 also includes modules for implementing short range telemetry 212 (“SR Telex”), long range telemetry 213 (“LR Telex”), security 214, and data processing 215. Depending upon the type of interfacing provided, the portable patient management device 211 can further include modules for implementing database management 216, data analysis 217, output management 218, and programming 219.

Short range telemetry 212 and long range telemetry 213 respectively implement telemetric interfaces for communicating with IMDs, as identified in a list of devices and monitors 225, and external devices, as identified in a list of programmers and external devices 226. Short range telemetry includes inductive, RF, and wireless telemetry, while long range telemetry 213 includes wired or wireless interfaces, such as “WiFi,” “WiMax,” and “strong” BlueTooth. Other types of short range and long range telemetry are possible. Security 214 handles authentication through use of the stored credentials 221 and provides primary and secondary security, such as encryption, decryption, certification, compression, and decompression, in concert with the exchange of patient data. In conjunction with data analysis 217, as further described below, data processing 215 can enable programmer-type functionality without built-in detailed output features. In addition, data processing 215 performs a core set of functions common to all interfacing, such as retrieving patient data from an IMD and storing the patient data 223 in the storage 220. Additionally, data processing 215 performs interrogation and data exchange respectively with the IMDs and external devices. Other types of core functionality can be provided.

Database manager 216, data analysis 217, and output manager 218 respectively perform the operations described above with reference to FIGS. 7-9. The operations required to output or relay unprocessed data 232 or processed data 233 to a programmer are generally provided by the modules providing short range telemetry 212, long range telemetry 213, security 214, and data processing 215. The database manager 216 converts and formats the physiological measures 230 that are received as patient data based on stored database formats 225 to generate database records 234. Similarly, data analysis 217 processes the physiological measures 230 based on stored data analysis programs 226 to provide processed data 233, which can be forwarded to the external devices 125-132 for output to primary or referring clinicians or to the patient. In a further embodiment, data analysis 217 can be tailored to a specific physician practice or specialty and can also be configured to generate a summary of patient information. Finally, the output manager 218 formats the physiological measures 230 based on stored hard copy formats 227 to provide reports and hard copy 235. In a further embodiment, programming 219 receives control parameters 231 from an external device or, in a still further embodiment, via a user interface provided by the portable patient management device 211 (not shown), that specify control profile changes that are provided to one or more IMDs 123 as programming parameters 236. Other types of portable patient management device operations are possible.

Portable Form Factor

In one embodiment, the portable patient management device is implemented in a convenient handheld and battery-operated form factor. FIG. 11 is a functional block diagram showing, by way of example, a portable patient management device 240 in handheld form factor 241, in accordance with one embodiment. The portable patient management device 240 implements some or all of the functionality described above with reference to FIG. 10. Preferably, the portable patient management device 240 can be manufactured as a lower-cost alternative to a conventional programmer and can be made available to both physicians and patients for in-clinic and at large use. The relative affordability and availability of the device makes patient data accessible faster than current methods available using, for instance, a conventional programmer, due to the user-friendly user interface and the ability to provide direct outputs to external devices, particularly hard copy devices

The handheld form factor 241 includes a user interface 242 that includes a plurality of user-operable buttons. Each button is preferably labeled with an icon or label identifying the function performed. For instance, to prepare the device for IMD interrogation, an “Interrogate Ready” button 243 can be pressed. Similarly, to prepare the device for interfacing with an external device, an “External Device Ready” button 244 can be pressed. Both IMD interrogation and external device interfacing are executed when a “Commit” button 246 is pressed. An operation can be canceled by pressing a “Cancel” button 245 and a downloaded set of patient data can be discarded by pressing a “Discard” button 247. User assistance can be provided by pressing a “Help” button 248. Other buttons and functionality can be provided.

While the invention has been particularly shown and described as referenced to the embodiments thereof, those skilled in the art will understand that the foregoing and other changes in form and detail may be made therein without departing from the spirit and scope of the invention.

What is claimed is:

1. A patient management device for portably interfacing with a plurality of implantable medical devices, comprising:
stored credentials to authenticate permission to interrogate one or more implantable medical devices;
a short range telemetry interface to individually exchange patient device data through interrogation of at least one authenticated implantable medical device;
a long range telemetry interface to exchange external device data via communication with at least one external device; and
storage to maintain at least one of the patient device and external device data contemporaneously to execution of operations to perform one or more of relay, processing, and outputting of the patient device and external device data subsequent to the interrogation of the implantable medical device.

2. A patient management device according to claim 1, further comprising:
a processor to perform at least one of processing at least one of the patient device and external device data to determine a tangible result and converting at least one of the patient device and external device data to effect a change in form or structure.

3. A patient management device according to claim 1, further comprising:
a database manager to perform at least one of converting and formatting at least one of the patient device and external device data into database records.

4. A patient management device according to claim 1, further comprising:
a data analyzer to perform at least one of statistical analysis, threshold evaluation, derivation, reduction, and extrapolation of at least one of the patient device and external device data.

5. A patient management device according to claim 1, further comprising:
an output manager to perform at least one of transformation, translation, and formatting of at least one of the patient device and external device data.

6. A patient management device according to claim 1, further comprising:
a programmer to program operation of at least one such implantable device.

7. A patient management device according to claim 1, further comprising:
security to perform at least one of encryption, decryption, certification, compression, and decompression in concert with the exchange of at least one of the patient device and external device data.

8. A patient management device according to claim 1, further comprising:
a user interface facilitating user configuration, operation, and programming of the exchange of at least one of the patient device and external device data.

9. A patient management device according to claim 1, further comprising:
further stored credentials to authenticate permission to communicate with the at least one external device.

10. A patient management device according to claim 1, wherein the short range telemetry interface is selected from the group comprising inductive, radio frequency, and wireless telemetry.

11. A patient management device according to claim 1, wherein the long range telemetry interface is selected from the group comprising wired, wireless, cellular, and telephonic network telemetric access.

12. A patient management device according to claim 1, wherein the external device is configured to perform a function selected from the group comprising programming, recording, data analysis, hard copy output, electronic output, database management, data relay, computation, and communication.

13. A patient management device according to claim 1, wherein at least one such implantable device is configured to perform a function selected from the group comprising pacing, cardiac resynchronization, defibrillation, neural stimulation and drug delivery, and physiological data monitoring.

14. A method for portably interfacing with a plurality of implantable medical devices, comprising:
authenticating permission to interrogate one or more implantable medical devices using stored credentials;
individually exchanging patient device data through interrogation of at least one authenticated implantable medical device through short range telemetry;
exchanging external device data via communication with at least one external device through long range telemetry; and
maintaining at least one of the patient device and external device data contemporaneously to execution of operations to perform one or more of relay, processing, and outputting of the patient device and external device data subsequent to the interrogation of the implantable medical device.

15. A method according to claim 14, further comprising:
performing at least one of:
processing at least one of the patient device and external device data to determine a tangible result; and
converting at least one of the patient device and external device data to effect a change in form or structure.

16. A method according to claim 14, further comprising:
performing at least one of converting and formatting at least one of the patient device and external device data into database records.

17. A method according to claim 14, further comprising:
performing at least one of statistical analysis, threshold evaluation, derivation, reduction, and extrapolation of at least one of the patient device and external device data.

18. A method according to claim 14, further comprising:
performing at least one of transformation, translation, and formatting of at least one of the patient device and external device data.

19. A method according to claim 14, further comprising:
programming operation of at least one such implantable device.

20. A method according to claim 14, further comprising:
performing at least one of encryption, decryption, certification, compression, and decompression in concert with the exchange of at least one of the patient device and external device data.

21. A method according to claim 14, further comprising:
facilitating user configuration, operation, and programming of the exchange of at least one of the patient device and external device data.

22. A method according to claim 14, further comprising:
authenticating permission to communicate with the at least one external device using further stored credentials.

23. A method according to claim 14, wherein the short range telemetry interface is selected from the group comprising inductive, radio frequency, and wireless telemetry.

24. A method according to claim 14, wherein the long range telemetry interface is selected from the group comprising wired, wireless, cellular, and telephonic network telemetric access.

25. A method according to claim 14, wherein the external device is configured to perform a function selected from the group comprising programming, recording, data analysis, hard copy output, electronic output, database management, data relay, computation, and communication.

26. A method according to claim 14, wherein at least one such implantable device is configured to perform a function selected from the group comprising pacing, cardiac resynchronization, defibrillation, neural stimulation and drug delivery, and physiological data monitoring.

27. A computer-readable storage medium holding code for performing the method according to claim 14.

28. An apparatus portably interfacing with a plurality of implantable medical devices, comprising:
means for authenticating permission to interrogate one or more implantable medical devices;
means for individually exchanging patient device data through interrogation of at least one implantable medical device through short range telemetry;
means for exchanging external device data via communication with at least one external device through long range telemetry; and
means for maintaining at least one of the patient device and external device data contemporaneously to execution of means for performing one or more of relay, processing, and outputting of the patient device and external device data subsequent to the interrogation of the implantable medical device.

29. A system for portably interrogating a plurality of implantable medical devices, comprising:
stored credentials to authenticate permission to interrogate one or more implantable medical devices;
a short range telemetry interface to individually exchange patient device data through interrogation of at least one authenticated implantable medical device; and
a processor to process the patient device contemporaneously to execution of operations to perform one or more of presentation, relay, processing, and outputting of the patient device subsequent to the interrogation of the authenticated implantable medical device.

30. A system according to claim 29, further comprising:
a data analyzer to perform threshold evaluation by enumerating notifications of thresholds being exceeded.

31. A system according to claim 29, further comprising:
an interface to provide the patient data to a device selected from the group comprising a built-in user interface, stationary patient management device, programmer, database, personal computer, printer, facsimile machine, and portal to an internetwork.

32. A method for portably interrogating a plurality of implantable medical devices, comprising:
authenticating permission to interrogate one or more implantable medical devices using stored credentials;
individually exchanging patient device data through interrogation of at least one authenticated implantable medical device through short range telemetry; and
processing the patient device contemporaneously to execution of operations to perform one or more of presentation, relay, processing, and outputting of the patient device subsequent to the interrogation of the authenticated implantable medical device.

33. A method according to claim 32, further comprising:
performing threshold evaluation by enumerating notifications of thresholds being exceeded.

34. A method according to claim 32, further comprising:
providing the patient data to a device selected from the group comprising a built-in user interface, stationary patient management device, programmer, database, personal computer, printer, facsimile machine, and portal to an internetwork.

35. A computer-readable storage medium holding code for performing the method according to claim 32.

36. An apparatus for portably interrogating a plurality of implantable medical devices, comprising:
means for authenticating permission to interrogate one or more implantable medical devices using stored credentials;
means for individually exchanging patient device data through interrogation of at least one authenticated implantable medical device through short range telemetry; and
means for processing the patient device contemporaneously to execution of operations to perform one or more of presentation, relay, processing, and outputting of the patient device subsequent to the interrogation of the authenticated implantable medical device.
